(19) United States
(12) Patent Application Publication
(10) Pub. No.: US 2015/0342610 A1
(43) Pub. Date: Dec. 3, 2015

(54) MEDICAL DEVICES AND METHODS FOR LUNG VOLUME REDUCTION

(71) Applicant: PulmonX Corporation, Redwood City, CA (US)

(72) Inventors: Sri Radhakrishnan, Cupertino, CA (US); Ryan Olivera, Granite Bay, CA (US); Hoang Nguyen, San Jose, CA (US)

(21) Appl. No.: 14/724,400

(22) Filed: May 28, 2015

Related U.S. Application Data
(60) Provisional application No. 62/004,377, filed on May 29, 2014.

(51) Int. Cl. A61B 17/12; A61B 17/128; A61B 17/122

(52) U.S. Cl. CPC A61B 17/12013; A61B 17/122; A61B 17/1285; A61B 17/12104; A61B 2017/00809

(57) ABSTRACT

An implantable device for reducing the volume of a lung compartment is disclosed. Aspects of the device includes a first contact element configured to contact with an inner wall of a first airway; a second contact element configured to contact with an inner wall of a second airway; and a compression element configured to apply a compressive force between the first and the second contact elements and to move the first contact element and the second contact element towards each other such that a space between the first airway and the second airway is compressed.

### CROSS-REFERENCE TO RELATED APPLICATION

This application claims priority under U.S.C. §119(e) to U.S. Provisional Patent application Ser. No. 62/004,377 (Attorney Docket No. 20920-772.101), entitled Medical Devices and Methods for Lung Volume Reduction, filed May 29, 2014, the full disclosure of which is incorporated herein by reference.

### FIELD OF THE INVENTION

This invention relates generally to medical devices and more specifically to devices, systems and methods for treating tissue using implants to achieve lung volume reduction by altering the airways of a lung region.

### BACKGROUND OF THE INVENTION

Chronic obstructive pulmonary disease (COPD) is a significant medical problem affecting 16 million people or about 6% of the U.S. population. Specific diseases in this group include chronic bronchitis, asthmatic bronchitis, and emphysema. While a number of therapeutic interventions are used and have been proposed, none are completely effective, and chronic obstructive pulmonary disease remains the fourth most common cause of death in the United States. Thus, improved and alternative treatments and therapies would be of significant benefit.

Of particular interest to the present invention, lung function in patients suffering from some forms of chronic obstructive pulmonary disease can be improved by reducing the effective lung volume, typically by resecting diseased portions of the lung. Resection of diseased portions of the lungs both promotes expansion of the non-diseased regions of the lung and decreases the portion of inhaled air which goes into the lungs but is unable to transfer oxygen to the blood. Lung volume reduction is conventionally performed in open chest or thoracoscopic procedures where the lung is resected, typically using stapling devices having integral cutting blades.

While effective in many cases, conventional lung volume reduction surgery (LVRS) is significantly traumatic to the patient, even when thoracoscopic procedures are employed. Such procedures often result in the unintentional removal of healthy lung tissue, and frequently leave perforations or other discontinuities in the lung which result in air leakage from the remaining lung. Even technically successful procedures can cause respiratory failure, pneumonia, and death. In addition, many older or compromised patients are not able to be candidates for these procedures.

As an alternative to LVRS, endobronchial lung volume reduction (ELVR) uses endobronchially introduced devices which plug or otherwise isolate a diseased compartment from healthier regions of the lung in order to achieve volume reduction of the diseased compartment. Isolation devices may be implanted in the main airways feeding the diseased region of the lung, and volume reduction takes place via absorption atelectasis after implantation or via collapse by actively suctioning of the target compartment prior to implantation. These implanted isolation devices can be, for example, self-expanding occlusive stents that prevent air flow in either directions, or one-way valves that allow flow in the exhalation direction only.

While a significant improvement over LVRS, ELVR can have a limited therapeutic benefit when the treated region in the lung is exposed to collateral ventilation from adjacent regions. The lungs comprise a plurality of compartments, referred to as lung compartments or lobes, which are separated from one another by a double layer of enfolded reflections of visceral pleura, referred to as fissures. While the fissures which separate the compartments are typically impermeable, in patients suffering from COPD, the fissures are frequently incomplete, leaving a pathway for collateral airflow or inter-lobular collateral ventilation. Such collateral airflow can result in the intrusion of air into the isolated lung compartments treated by ELVR, thus reducing or eliminating the desired volume reduction.

For these reasons, it would be desirable to provide alternative and improved methods and apparatus for lung volume reduction. At least some of these objectives will be met by the inventions described herein below.

### BRIEF SUMMARY OF THE INVENTION

In some aspects, the present application discloses methods, systems, and devices for reducing the volume of a lung compartment.

In one aspect, a device for reducing the volume of a lung compartment comprises a first contact element configured to contact with an inner wall of a first airway; a second contact element configured to contact with an inner wall of a second airway; and a compression element configured to apply a compressive force between the first and the second contact elements and to move the first contact element and the second contact element towards each other such that a space between the first airway and the second airway is compressed or reduced.

These and other aspects of the present disclosure are described herein.

### BRIEF DESCRIPTION OF THE DRAWINGS

Present embodiments have other advantages and features which will be more readily apparent from the following detailed description and the appended claims, when taken in conjunction with the accompanying drawings, in which:

FIG. 1A illustrates an anterior view of a pair of human lungs and a bronchial tree.

FIG. 1B illustrates a lateral view of the right lung.

FIG. 1C illustrates a lateral view of the left lung.

FIG. 1D illustrates an anterior view of the trachea and a portion of the bronchial tree.

FIG. 2A illustrates one embodiment of the device with two contact elements connected by a compression element.

FIG. 2B illustrates exemplary airways where one embodiment of the compression device may be placed.

FIG. 2C illustrates one embodiment of the compression device as illustrated in FIG. 2A placed in the airways shown in FIG. 2B.

FIG. 3 illustrates one embodiment of the compression device with two contact elements connected by a compression element comprising a triangular joint.

FIG. 4 illustrates one embodiment of the compression device comprising magnetic elements.

FIG. 5A illustrates one embodiment of the compression device comprising two separate contact elements where the contact elements comprise magnetic compression elements.

FIG. 5B illustrates exemplary airways where the compression device may be placed.

FIG. 5C illustrates one embodiment of the compression device as illustrated in FIG. 5A placed in the airways illustrated in FIG. 5B.

FIG. 6A illustrates an embodiment of a compression device in an expanded state comprising two contact elements and a separate compression element.

FIG. 6B illustrates an embodiment of a compression device in a compressed state comprising two contact elements and a separate compression element.

FIG. 7A illustrates an embodiment of a compression device in an expanded state comprising two contact elements with at least one locking element and a separate compression element.

FIG. 7B illustrates an embodiment of a compression device in a compressed state comprising two contact elements with locking elements and a separate compression element.

FIG. 8A illustrates an embodiment of a compression device in an expanded state comprising three contact elements and a separate compression element.

FIG. 8B illustrates an embodiment of a compression device in a compressed state comprising three contact elements and a separate compression element.

FIG. 9A illustrates one embodiment of the compression device with two contact elements with curved distal portions.

FIG. 9B illustrates another embodiment of the compression device with two contact elements with curved proximal portions.

FIG. 10A illustrate an embodiment of the compression device with two contact elements with joint sections.

FIG. 10B illustrate another embodiment of the compression device with two contact elements with joint sections.

FIG. 10C illustrate yet another embodiment of the compression device with two contact elements with joint sections.

FIG. 11 illustrates an embodiment of the compression device where the contact elements comprise telescoping portions.

FIG. 12 illustrates another embodiment of the compression device with two contact elements comprising stabilization elements configured as barbs.

FIG. 13 illustrates another embodiment of the compression device with two contact elements comprising stabilization elements configured as a stent.

### DETAILED DESCRIPTION OF THE INVENTION

Although the detailed description contains many specifics, these should not be construed as limiting the scope of the disclosure but merely as illustrating different examples and aspects of the disclosure. It should be appreciated that the scope of the disclosure includes other aspects and embodiments not discussed herein. Various other modifications, changes and variations which will be apparent to those skilled in the art may be made in the arrangement, operation and details of the method, device, and system of the aspects and embodiments disclosed herein without departing from the spirit and scope of the disclosure as described here.

Throughout the specification and claims, the following terms take the meanings explicitly associated herein unless the context clearly dictates otherwise. The meaning of “a”, “an”, and “the” include plural references. The meaning of “in” includes “in” and “on.” Referring to the drawings, like numbers indicate like parts throughout the views. Additionally, a reference to the singular includes a reference to the plural unless otherwise stated or inconsistent with the disclosure herein.

The word “exemplary” is used herein to mean “serving as an example, instance, or illustration.” Any implementation described herein as “exemplary” is not necessarily to be construed as advantageous over other implementations.

Throughout this disclosure, reference is made to the term: “implantable device.” As used herein, the term “implantable device” refers to various implantable devices configured to be capable of being placed within a lung region to treat pulmonary disorders. In some aspects, the term “implantable device” refers to implantable devices configured to alter an airway. Furthermore, the implantable device may be a device to treat vascular, urinary, biliary, esophageal, and renal tracts and the like.

Throughout this disclosure, reference is made to the term “lung region”. As used herein, the term “lung region” refers to a defined division or portion of a lung. For purposes of example, lung regions are described herein with reference to human lungs, wherein some exemplary lung regions include lung lobes and lung segments. Thus, the term “lung region” as used herein can refer, for example, to a lung lobe or a lung segment. Such nomenclature conforms to nomenclature for portions of the lungs that are known to those skilled in the art. However, it should be appreciated that the term “lung region” does not necessarily refer to a lung lobe or a lung segment, but can refer to some other defined division or portion of a human or non-human lung.

Throughout this disclosure, reference is made to the term “airways.” As used herein, the term “airways” refers to the airway passages that transmit air from the atmosphere to the alveoli. For purposes of example, airways are described herein with reference to human lungs, wherein some exemplary lung regions include bronchi and bronchioles. Thus, the term “airways” as used herein can refer, for example, to the bronchi or bronchioles.

Present disclosure describes systems, devices, and methods to achieve or to maintain lung volume reduction, and more specifically to systems, devices, and methods for treating a lung region by using implants to achieve or to maintain lung volume reduction by altering one or more airways of a lung region. In some aspects, present disclosure describes embodiments of implantable devices configured to alter or modify the airways such that a plurality of airways are pulled together thus reducing the space between the airways resulting in lung volume reduction.

Throughout this description, certain terms are used that refer to relative directions or locations along a path defined from an entryway into the patient's body (e.g., the mouth or nose) to the patient's lungs. The path of airflow into the lungs generally begins at the patient's mouth or nose, travels through the trachea into one or more bronchial passageways, and terminates at some point in the patient's lungs.

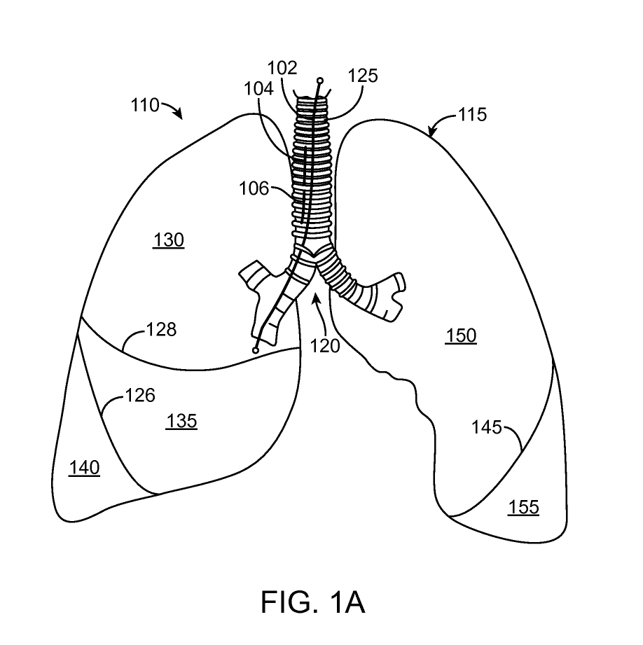

For example, FIG. 1A shows a path 102 that travels through the trachea 125 and through a bronchial passageway into a location in the right lung 110. The term “proximal direction” refers to the direction along such a path 102 that points toward the patient's mouth or nose and away from the patient's lungs. In other words, the proximal direction is generally the same as the expiration direction when the patient breathes. The arrow 104 in FIG. 1A points in the proximal or expiratory direction. The term “distal direction” refers to the direction along such a path 102 that points toward the patient's lung and away from the mouth or nose. The distal direction is generally the same as the inhalation or inspiratory direction when the patient breathes. The arrow 106 in FIG. 1A points in the distal or inhalation direction.

The lungs include a right lung 110 and a left lung 115. The right lung 110 includes lung regions comprised of three lobes, including a right upper lobe 130, a right middle lobe 135, and a right lower lobe 140. The lobes 130, 135, 140 are separated by two interlobar fissures, including a right oblique fissure 126 and a right transverse fissure 128. The right oblique fissure 126 separates the right lower lobe 140 from the right upper lobe 130 and from the right middle lobe 135. The right transverse fissure 128 separates the right upper lobe 130 from the right middle lobe 135.

As shown in FIG. 1A, the left lung 115 includes lung regions comprised of two lobes, including the left upper lobe 150 and the left lower lobe 155. An interlobar fissure comprised of a left oblique fissure 145 of the left lung 115 separates the left upper lobe 150 from the left lower lobe 155. The lobes 130, 135, 140, 150, 155 are directly supplied air via respective lobar bronchi, as described in detail below.

FIG. 1B is a lateral view of the right lung 110. The right lung 110 is subdivided into lung regions comprised of a plurality of bronchopulmonary segments. Each bronchopulmonary segment is directly supplied air by a corresponding segmental tertiary bronchus, as described below. The bronchopulmonary segments of the right lung 110 include a right apical segment 210, a right posterior segment 220, and a right anterior segment 330, all of which are disposed in the right upper lobe 130. The right lung bronchopulmonary segments further include a right lateral segment 240 and a right medial segment 250, which are disposed in the right middle lobe 135. The right lower lobe 140 includes bronchopulmonary segments comprised of a right superior segment 260, a right medial basal segment (which cannot be seen from the lateral view and is not shown in FIG. 1B), a right anterior basal segment 280, a right lateral basal segment 290, and a right posterior basal segment 295.

FIG. 1C shows a lateral view of the left lung 115, which is subdivided into lung regions comprised of a plurality of bronchopulmonary segments. The bronchopulmonary segments include a left apical segment 310, a left posterior segment 320, a left anterior segment 330, a left superior segment 340, and a left inferior segment 350, which are disposed in the left lung upper lobe 150. The lower lobe 155 of the left lung 115 includes bronchopulmonary segments comprised of a left superior segment 360, a left medial basal segment (which cannot be seen from the lateral view and is not shown in FIG. 1C), a left anterior basal segment 380, a left lateral basal segment 390, and a left posterior basal segment 395.

FIG. 1D shows an anterior view of the trachea 125 and a portion of the bronchial tree 120, which includes a network of bronchial passageways, as described below. The trachea 125 divides at a lower end into two bronchial passageways comprised of primary bronchi, including a right primary bronchus 410 that provides direct air flow to the right lung 110, and a left primary bronchus 415 that provides direct air flow to the left lung 115. The ridge-like structure known herein as the carina is formed as a downward and backward projection of the lowest tracheal cartilage or as a ridge between the openings of the right and left principal bronchi. As used herein, carina is used to refer to any structures forming a projecting central ridge between airways, including, but not limited to the principal bronchi. Each primary bronchus 410, 415, divides into a next generation of bronchial passageways comprised of a plurality of lobar bronchi. The right primary bronchus 410 divides into a right upper lobar bronchus 417, a right middle lobar bronchus 420, and a right lower lobar bronchus 422. The left primary bronchus 415 divides into a left upper lobar bronchus 425 and a left lower lobar bronchus 430.

Each lobar bronchus 417, 420, 422, 425, 430 directly feeds fluid to a respective lung lobe, as indicated by the respective names of the lobar bronchi. The lobar bronchi each divides into yet another generation of bronchial passageways comprised of segmental bronchi, which provide air flow to the bronchopulmonary segments discussed above.

As is known to those skilled in the art, a bronchial passageway defines an internal lumen through which fluid can flow to and from a lung or lung region. The diameter of the internal lumen for a specific bronchial passageway can vary based on the bronchial passageway's location in the bronchial tree (such as whether the bronchial passageway is a lobar bronchus or a segmental bronchus) and can also vary from patient to patient. However, the internal diameter of a bronchial passageway is generally in the range of 3 millimeters (mm) to 10 mm, although the internal diameter of a bronchial passageway can be outside of this range. For example, a bronchial passageway can have an internal diameter of well below 1 mm at locations deep within the lung. The internal diameter can also vary from inhalation to exhalation as the diameter increases during inhalation as the lungs expand, and decreases during exhalation as the lungs contract.

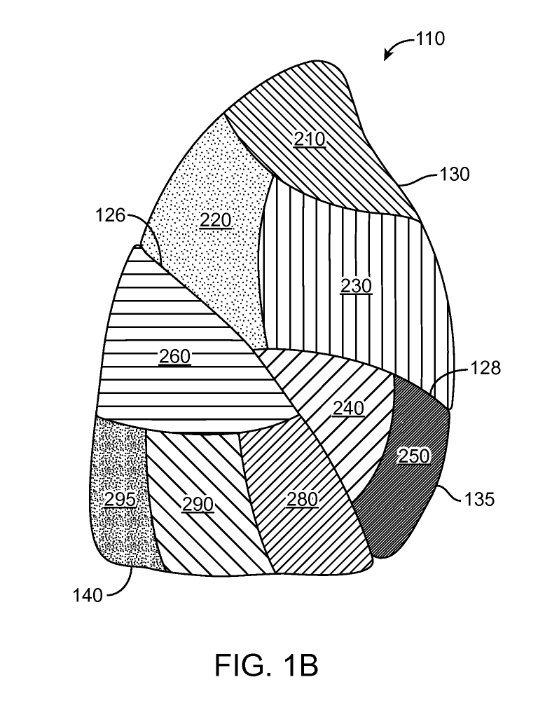

Referring now to FIG. 2A, where one embodiment of the present disclosure is exemplarily shown. As seen in FIG. 2A, one aspect of the compression device 500 comprises a first contact element 510 and a second contact element 520 configured to engage or to contact with a portion of an airway. In some aspects, at least one of the two contact elements 510, 520, comprises an elongated contacting portion configured with a surface capable of engaging with a portion of the airway. It is contemplated that the contacting portion may assume various curvatures or dimensions to stabilize and secure the contact element within the airways. It is further contemplated that the contacting portion may comprise various stabilization elements such as ridges, barbs or, additionally or alternatively, the contacting portion may be configured with textures to stabilize and to maintain the contact between the contact elements and the airways.

As seen in FIG. 2A, the two contact elements 510, 520 are connected by a compression element 530. In some aspects, and as exemplarily shown, the compression element 530 is configured as a substantially rounded joint connecting the two contact elements. In one embodiment, the compression element 530 is configured to impart a compressive force to cause the first contact element 510 and the second contact element 520 to compress from an expanded state. In one embodiment as shown in FIG. 2A, where the compression element 530 is configured as a rounded joint connecting the two contact elements, the compression element 530 is configured to impart a spring force as the compressive force, where the spring force transferred to the contact elements 510, 520 to transform the contact elements 510, 520 from an expanded state to a compressed state.

Additionally and optionally, one or both of the contact elements 510, 520 may comprise one or more atraumatic portions configured to reduce tissue trauma once the contact elements have engaged with tissue. In some aspects, as seen in FIG. 2A, the contact elements comprise a pair of atraumatic tips 511, 521 at the distal portion of each contact element 510, 520.

Referring now to FIGS. 2B and 2C, FIG. 2B illustrates exemplary airways where an embodiment of the device may be inserted and FIG. 2C illustrates the effect to the airways after the placement of the device. After inserting the device into the lung compartment and positioning the device near the target airways, the device is placed at the carina C formed at a branch of first and second airway, A1, A2. The first contact element 510 is placed in contact with an inner wall of the first airway A1 and the second contact element 520 is placed in contact with an inner wall of the second airway A2. In one aspect, to insert the device at the carina C, the two contact elements 510, 520 are transformed to an expanded configuration by applying mechanical forces to one or both of the contact elements 510, 520. In one aspect, the compression device 500 in the expanded state is then placed over the carina C. In one embodiment, the tip of the carina C is positioned between the rounded joint to secure the compression device 500 to the carina C. Thereafter, in one aspect, once the compression device 500 is placed at the carina C, the force of the compression element 530 provides a compressive force to bring the first contact element 510 and the second contact element 520 to the compressed state, thus compressing the carina C. The compression of carina C reduces the space between the airways and thus brings the first airway A1 and the second airway A2 closer together, which results in reduction of the lung volume.

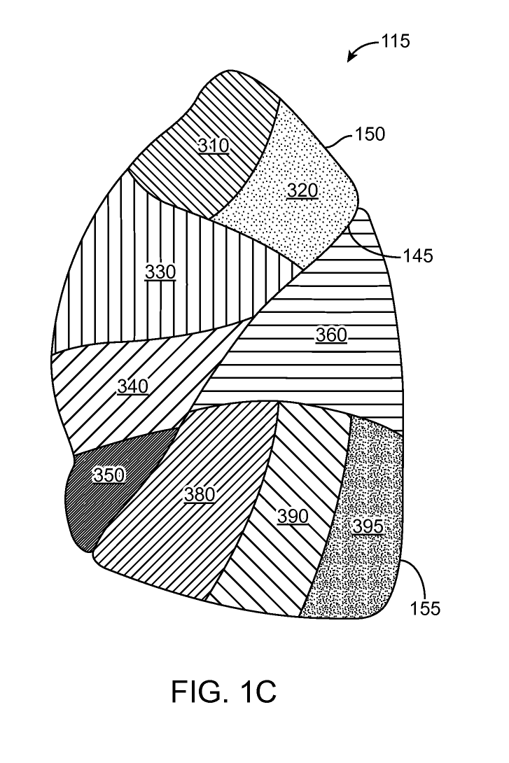

FIG. 3 shows another embodiment where the compression device 600 comprises two contact elements 610, 620 joined together by a compression element 630 configured as a triangular joint. In one embodiment, the apex 631 of the triangular joint is connected to the two contact elements 610, 620. Additionally and optionally, one or both of the contact elements 610, 620 may comprise one or more atraumatic portions configured to reduce tissue trauma once the contact elements have engaged with tissue. In some aspects, as seen in FIG. 3, the contact elements comprise a pair of atraumatic tips 611, 621 at the distal portion of each contact element 610, 620.

In one application of the compression device 600 as exemplarily shown in FIG. 3, when a force is applied to the contact elements to move the contact elements 610, 620 away from each other, the triangular joint is transformed to an expanded state from an initial configuration such that the base 632 of the triangle is deformed while the apex 631 of the triangle is pulled apart thus creating a separation space. When the force as applied to the contact elements is eliminated, the base 632 of the triangle returns to the initial configuration while the separation space at the apex 631 is reduced or eliminated. When operating this embodiment within the lung, after inserting the compression device 600 into the body and positioning the device near the target airways, the compression element 630 configured as a triangular joint is transformed to an expanded configuration. Thereafter, a portion of the carina formed at a branch of a first airway and a second airway is placed within the separation space at the apex while the first and the second contact elements 610, 620 engage with the inner walls of the airways. Thereafter, the compression element 630 configured as a triangular joint returns to the initial configuration such that the separation space at the apex 631 is reduced, thus compressing a portion of the carina within the apex. Additionally, as the compression element 630 returns to the initial configuration, the two contact elements 610, 620 are also moved towards each other, thus further compressing the airways. Alternatively, in one embodiment, a portion of the carina is not inserted into the separation space of the apex 631, instead, in this embodiment, compression is achieved between the contact elements 610, 620.

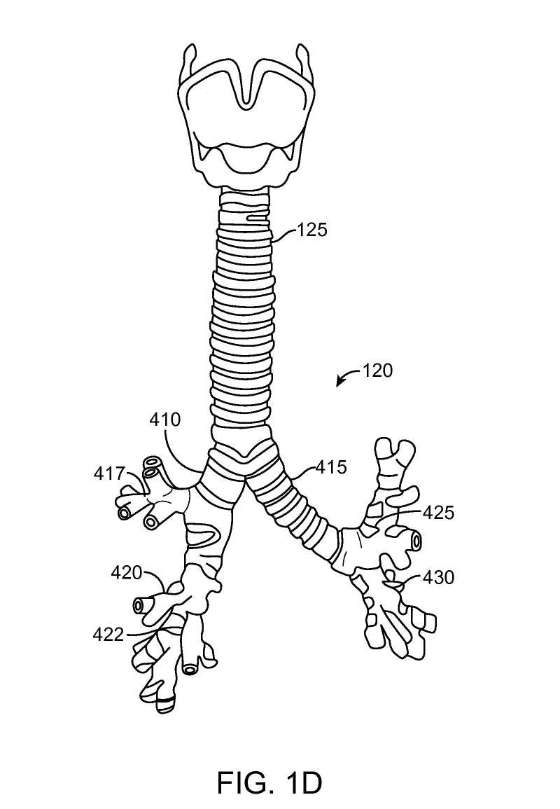

Referring now to FIG. 4, in another embodiment, the compression device 700 comprises two contact elements 710, 720 joined together by a compression element 730 exemplarily shown as circular joint. The two contact elements 710, 720 each comprise at least one magnetic element 740, 750. The magnetic elements 740, 750 are configured to produce magnetic fields such that the two contact elements 710, 720 are attracted to each other. In one embodiment, the magnetic elements 740, 750 comprise ferromagnetic material such as iron, nickel, cobalt, rare earth metals, etc. In some aspects, the magnetic elements 740, 750 are configured as a second compression element where the magnetic force produced by the magnetic elements 740, 750 creates a compression force. In some aspects, the second compression element acts in conjunction with the compression element 730 to either cause the contact elements 710, 720 to achieve a compressed state (where the contact elements are brought together) or to maintain a compressed state. Alternatively, in one embodiment of the device, only one of the contact elements 740 or 750 comprises a magnetic element, whereas the other contact element comprises material that can be affected by the magnetic element.

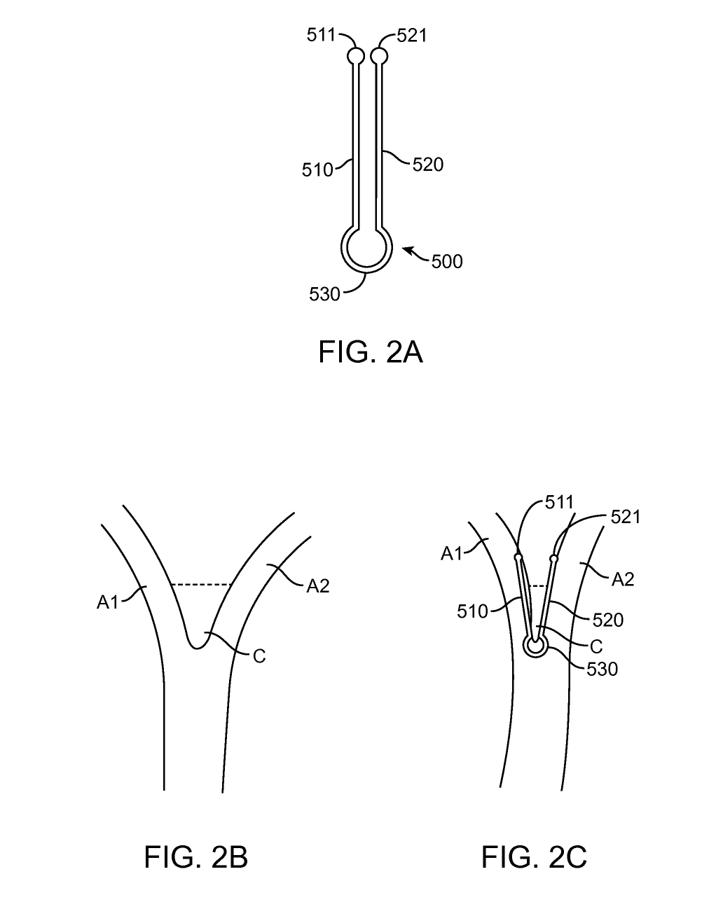

Referring now to FIG. 5A, in another embodiment, the compression device 800 comprises two separate contact elements 810, 820, where at least one of the contact elements 810, 820 comprises a magnetic compression element configured to impart a compressive force between the two contact elements 810, 820. Additionally and optionally, one or both of the contact elements may comprise one or more atraumatic portions configured to reduce tissue trauma once the contact elements have engaged with tissue. In some aspects, as seen in FIG. 5A, the contact elements each comprises a pair of atraumatic tips 811A, 811B and 821A, 821B at the distal portion and the proximal portion of each contact element 810, 820.

In some aspects, one of the contact elements comprises the magnetic compression element, while in other aspects, each of the contact elements 810, 820 comprises a magnetic compression element. In this embodiment, the magnetic compression element is configured to produce magnetic fields such that the two contact elements are attracted to each other, thus achieving a compressed state. In one embodiment, the magnetic elements comprise ferromagnetic material such as iron, nickel, cobalt, rare earth metals, etc.

The magnetic compression element creates a compressive force to either cause the contact elements to achieve a compressed state (where the contact elements are brought together) or to maintain a compressed state. Alternatively, in one embodiment of the device, only one of the contact elements comprises a magnetic compression element, whereas the other contact element comprises material that can be affected by the magnetic force created by the compression element.

Referring now to FIGS. 5B and 5C, FIG. 5B illustrates exemplary airways where an embodiment of the device may be inserted and FIG. 5C illustrates the effect to the airways after the placement of the device. After inserting the compression device into the lung compartment and positioning the compression device 800 near the target airways, the device is placed around the carina C formed at a branch of a first and second airway A1, A2. The first contact element 810 is placed in contact with an inner wall of the first airway A1 and the second contact element 820 is placed in contact with an inner wall of the second airway A2. Once the two contact elements are placed around the carina C, the magnetic force of the compression element provides a compressive force to bring the first contact element 810 and the second contact element 820 to the compressed state, thus compressing the carina C. The compression of the carina reduces the space between the airways and thus brings the first airway A1 and the second airway A2 closer together, resulting in reduction to the lung volume.

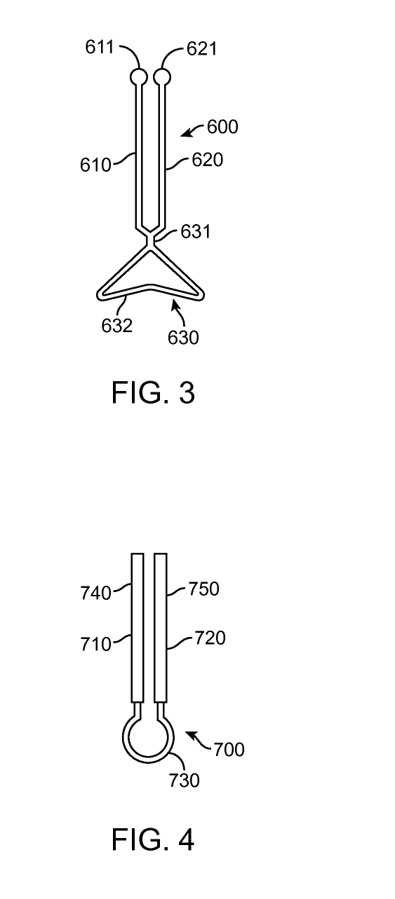

Referring now to FIGS. 6A-6B, in another embodiment, compression device 900 comprises a first portion comprising two body sections configured as first contact element 910 and second contact element 920. The device further comprises a separate compression element 930. Additionally and optionally, one or both of the contact elements 910, 920 may comprise one or more atraumatic portions configured to reduce tissue trauma once the contact elements have engaged with tissue. In some aspects, as seen in FIGS. 6A-6B, the contact elements comprise a pair of atraumatic tips 911, 921 at the distal portion of each contact element 910, 920.

In some aspects, the compression element 930 is configured as a sleeve capable of covering a proximal portion of the two contact elements 910, 920. The compression element 930 may be an elastic sleeve such that it may be first transformed to an expanded state to enable the compression element 930 to be placed over the first and the second contact elements 910, 920. Thereafter, the compression element 930 is transformed to a compressed state. In the compressed state, the compression element 930 is configured to impart compressive force to the two contact elements 910, 920.

In another embodiment, the compression element 930 may be a hypotube that is inserted over the contact elements 910, 920. After the insertion, the compression element 930 is crimped, thus transforming the compression element 930 to a compressed state.

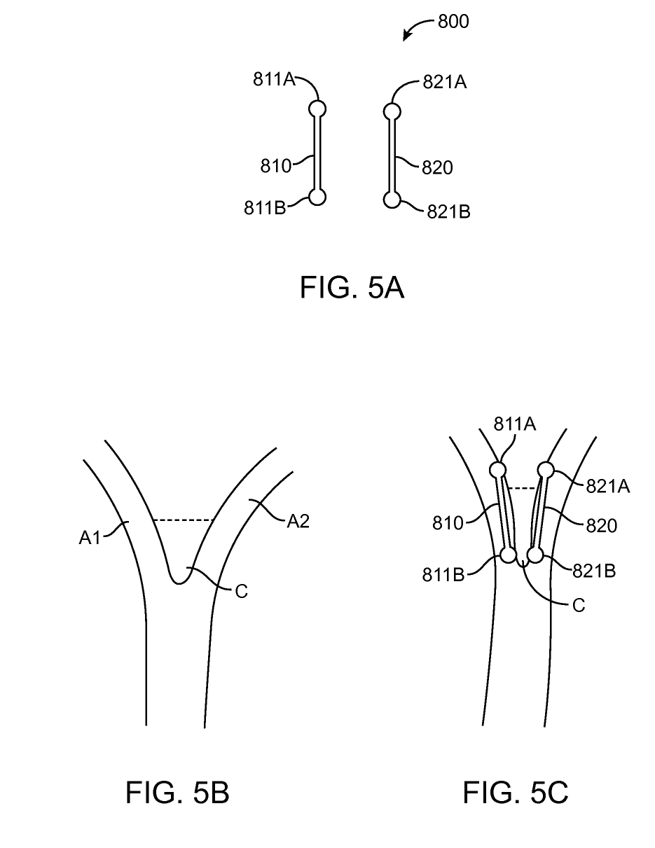

In some aspects of the present disclosure where the contact elements and the compression element are discrete units, the contact elements or the compression elements may comprise one or more locking elements configured to maintain the connection between the two elements. As illustrated in FIGS. 7A and 7B, an embodiment of the compression device 1000 comprises a first portion comprising two body sections configured as the first contact element 1010 and the second contact element 1020. In some aspects, as seen in FIGS. 7A and 7B, the contact elements comprise a pair of atraumatic tips 1011, 1021 at the distal portion of each contact element 1010, 1020. The device further comprises a separate compression element 1030. The compression element 1030 is configured as a sleeve capable of covering a proximal portion of the two contact elements 1010, 1020. The compression element 1030 is configured such that it is placed over the two contact elements 1010, 1020 in an expanded state, and thereafter transformed to a compressed state. In the compressed state, the compression element 1030 is configured to impart compression force to the two contact elements 1010, 1020.

As seen in FIGS. 7A and 7B, in one embodiment, the compression device 1000 comprises a locking element 1040 disposed on the two contact elements 1010, 1020. In one embodiment, where the compression element and the contact elements are discrete units, the locking element 1040 is configured to maintain connection of the compression element 1030 to the contact elements 1010, 1020 once the compression element 1030 has engaged with the contact elements 1010, 1020. In one embodiment, the locking element 1040 comprises two protrusions angled towards the proximal end of the contact elements such that they enable one directional movement over the protrusions, e.g., towards the proximal end of the contact elements 1010, 1020, while preventing movement in the opposite direction.

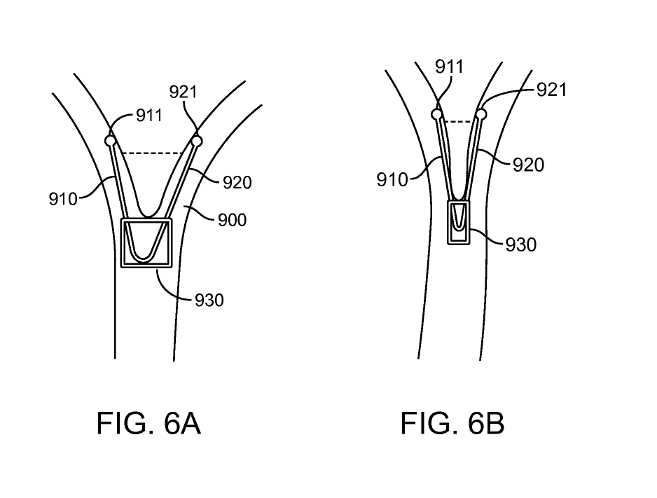

It is further contemplated that more than two contact elements may be used, this may be advantageous to concurrently achieve compression of multi-branched airways, where each of the contact elements is configured to contact with a portion of one of the airways. As seen in FIGS. 8A and 8B, a compression device 1100 comprises three individual contact elements 1110, 1120, 1130. In some aspects, as seen in FIGS. 8A and 8B, the contact elements comprise atraumatic tips 1111, 1121, 1131 at the distal portions of each contact element.

Each of the three contact elements as shown in FIGS. 8A and 8B is configured to be extended into and be in contact with a portion of an individual airway. In some aspects, the three contact elements are compressed using a single compression element 1140, where the compression element 1140 is a sleeve that is configured to be positioned over all three contact elements. In an alternative embodiment, multiple compression elements may be used. For example, in some aspects, the first and the second contact elements 1110, 1120 are in contact with a first compression element and the second and third contact elements 1120, 1130 are in contact with a second compression element, or various combinations thereof.

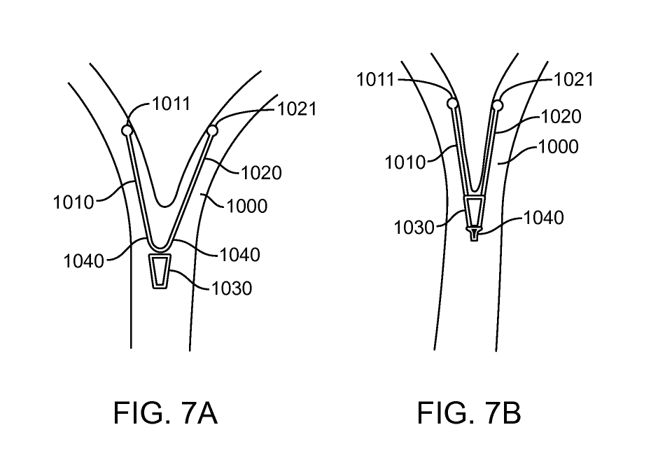

It is further contemplated that the contact elements as described in various embodiments may assume various curvatures or dimensions to stabilize or to secure the device within the airways or to facilitate the placement of the contact elements within the airway. For example, as shown in FIG. 9A, in one embodiment, a compression device 1200 comprises two contact elements 1210, 1220 connected by a compression element 1230. The two contact elements are configured to assume a curvature such that the proximal portions 1211, 1221 of the contact elements flare away from each other to facilitate placement and deployment of the device in airways. Additionally and optionally, as shown in FIG. 9B, the contact elements 1210, 1220 are configured to assume a curvature such that the distal portions 1212, 1222 of the contact elements flare away from each other to facilitate placement and deployment of the device in airways.

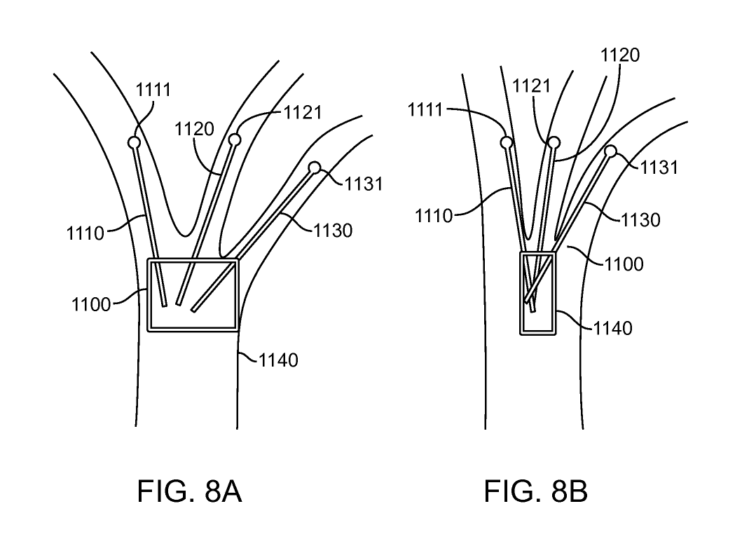

Additionally, as seen in FIGS. 10A-C, in some aspects the compression device 1300 comprises contact elements 1310, 1320 connected by a compression element 1330. The contact elements 1310, 1320 comprise joint sections 1311, 1321 that create additional points of compression and/or contact with the airways. As seen in the exemplary variations shown in FIGS. 10A-C, the joints may be placed at the distal portions of the contact elements 1310, 1320 or at the middle portions of the contact elements, or otherwise along the length of the contact elements. In some aspects, the joint sections 1311, 1321 are configured as semi-circular sections. In some aspects, the joint sections may advantageously be provided additional points of flexibility to the contact elements configured to facilitate the placement and insertion of the contact elements into airways with various lengths and shapes.

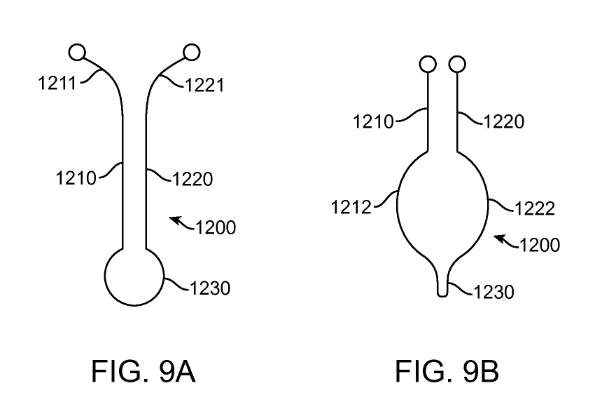

Referring now to FIG. 11, in one embodiment, a compression device 1400 comprises contact elements 1410, 1420 connected by a compression element 1430. The contact elements 1410, 1420 comprise telescoping portions 1440, 1450 that are configured to extend away from the distal portions of the contact elements 1410, 1420. The telescoping portions 1440, 1450 are configured to enable additional lengths of contact between the contact elements 1410, 1420 and the airways. In one exemplary operation, the compression device 1400 is inserted into body and placed at the target airways. Upon placement, the telescoping portions 1440, 1450 are configured to be extended further into the airways to stabilize the device after contact with the airways.

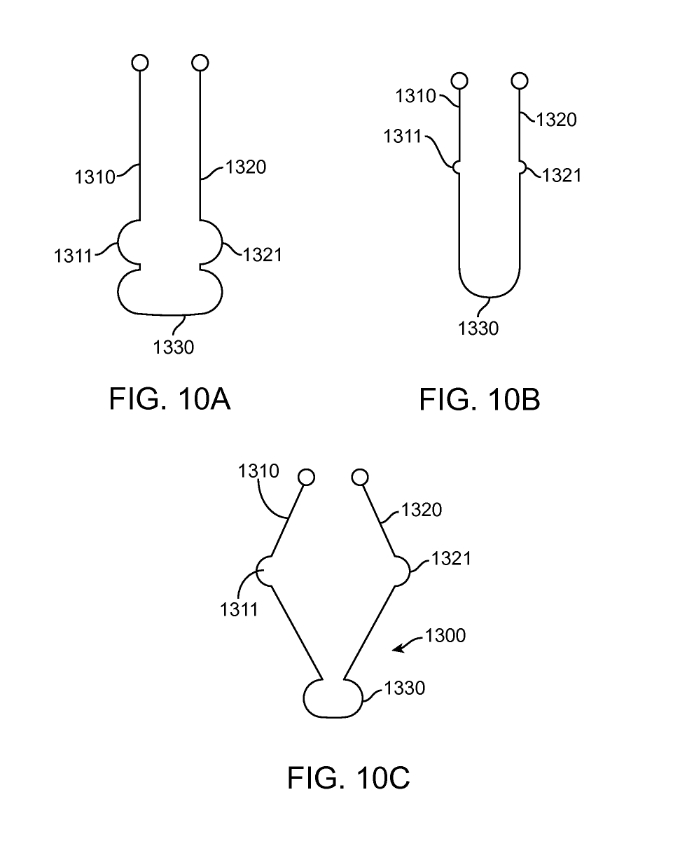

Various other stabilization elements may be employed to reinforce the connection between the contact elements and the airways and to assist in maintaining the position of the contact elements within the airways. Referring now to FIG. 12, where one embodiment of the compression device 1500 is shown. The device 1500 comprises two contact elements 1510, 1520 connected by a compression element 1530, where the contact elements 1510, 1520 further comprise stabilization elements 1540 exemplarily shown as barbs to prevent the device from moving once the contact elements 1510, 1520 engage with the airways. In some aspects, the barbs create semi-permanent connections between the contact elements 1510, 1520 and the tissues of the airways to further secure the device 1500 to the airways for long term lung volume reduction.

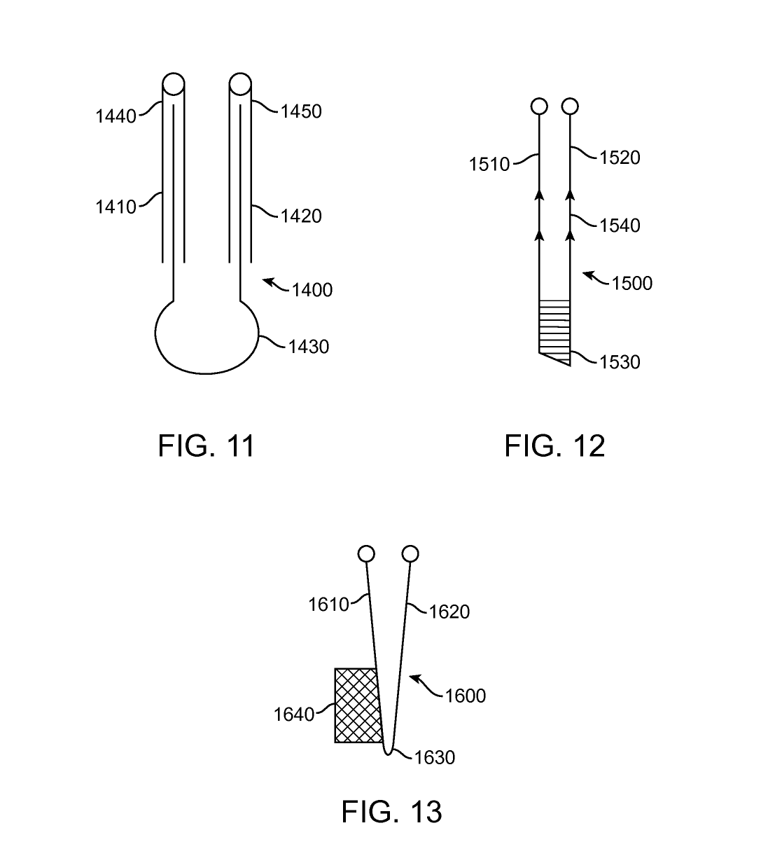

In an alternative embodiment, the stabilization element is configured as an expandable element that is connected to a contact element. Referring now to FIG. 13, the compression device 1600 comprises two contact elements 1610, 1620 with a stabilization element configured as a stent 1640 connected to the first contact element 1610. As seen in FIG. 13, the two contact elements 1610, 1620 are joined together by a compression element 1630 exemplarily shown as a rounded joint at the proximal portion of the device 1600. The compression device 1600 is first inserted into the body with the stent 1640 in a compressed state, after inserting the compression device 1600 into the lung region and that first and the second contact elements 1610, 1620 are in contact with the target airways. In some aspects, the stent 1640 is deployed using known methods to an expanded state to anchor the first contact element 1610 in the airway.

In the various embodiments described above, various parts of the device may be constructed of shape-memory materials including alloys or polymers, such as nitinol or poly(D,L-lactide), and are compressed to enable delivery through relatively small and curved bodily pathways to the lung region. In one embodiment, delivery devices, such as catheters, retain the collapsed pulmonary implants in a radially compressed state for delivery to the treatment site, where the implant is released into the lung region and regains its non-compressed shape.

It is further contemplated that the various embodiments described above may be implanted and removed or permanently implanted. It is also contemplated that the various embodiments described above may be bioabsorbable.

While the above is a complete description of various embodiments, any of a number of alternatives, modifications, and equivalents may be used in alternative embodiments. Therefore, the above description should not be taken as limiting the scope of the invention as it is defined by the appended claims.

In addition to above-mentioned components, the subject systems or kits comprising the described systems typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided. An example of this embodiment is a kit that includes a web address where the instructions can be viewed and/or from which the instructions can be downloaded. As with the instructions, this means for obtaining the instructions is recorded on a suitable substrate.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

What is claimed is:

1. An implantable device for reducing the volume of a lung compartment, said device comprising:
a first contact element configured to contact with an inner wall of a first airway;
a second contact element configured to contact with an inner wall of a second airway; and
a compression element configured to apply a compressive force between the first and the second contact elements and to move the first contact element and the second contact element towards each other such that a space between the first airway and the second airway is compressed.

2. The device of claim 1, wherein the first contact element, the second contact element and the compression element are connected.

3. The device of claim 2, wherein the compression element is a rounded joint connecting the first and the second contact elements.

4. The device of claim 2, wherein the compression element is a triangular joint connecting the first and the second contact elements.

5. The device of claim 2, wherein the compression element is configured to impart a tension by applying a spring force.

6. The device of claim 2, wherein the compression element comprises a first portion and a second portion, wherein the first portion and the second portion are configured to be biased towards each other.

7. The device of claim 1, wherein the compression element is a magnetic element connected to the first contact element.

8. The device of claim 7, further comprising a second compression element, wherein the second compression element is a second magnetic element connected to the second contact element.

9. The device of claim 8, wherein the compression element is configured to impart a tension by applying magnetic force on the second compression element.

10. The device of claim 1, wherein the compression element is a sleeve configured to cover a proximal portion of the first contact element and a proximal portion of the second contact element.

11. The device of claim 10, wherein the sleeve has a compressed configuration and an expanded configuration, and wherein the compressed configuration is configured to impart a tension to the contact elements.

12. The device of claim 10, wherein the first and second contact elements comprise locking elements configured to fix the sleeve in place.

13. The device of claim 1, further comprising a third contact element configured to contact with an inner wall of a third airway; and
wherein the compression element is further configured to impart a tension between the third contact element and the second contact element and to move the first, second, and third contact elements towards each other such that a space between the third airway and the second airway is compressed.

14. The device of claim 1, wherein the first contact element comprises a joint between a proximal portion and a distal portion of the first contact element, and the second contact element comprises a joint between a proximal portion and a distal portion of the second contact element.

15. The device of claim 1, wherein the first contact element and the second contact element are configured to have extendable lengths.

16. The device of claim 1, wherein the first contact element comprises an atraumatic tip at a distal portion of the first contact element, and the second contact element comprises an atraumatic tip at a distal portion of the second contact element.

17. The device of claim 5, further comprising a second compression element.

18. The device of claim 17, wherein the second compression element is a magnetic element connected to the first contact element.

19. The device of claim 1, wherein the first or second contact element comprises a flared tip at a distal end.

20. A method for reducing the volume of a lung compartment, said method comprising:
inserting a device comprising a first contact element, a second contact element and a compression element at a branch between a first airway and a second airway;
placing the first contact element in contact with an inner wall of the first airway; and
placing the second contact element in contact with an inner wall of the second airway;
wherein the compression element is configured to move the first contact element and the second contact element towards each other such that a space between the first airway and the second airway is compressed.

21. The method of claim 20, further comprising:
placing a third contact element in contact with an inner wall of a third airway; and
moving the third contact element and the second contact element towards each other using the compression element such that a space between the third airway and the second airway is compressed.

22. A method for reducing the volume of a lung compartment, said method comprising:
inserting a device comprising a first contact element, a second contact element and a compression element at a branch between a first airway and a second airway;
placing the first contact element in contact with an inner wall of the first airway; and
placing the second contact element in contact with an inner wall of the second airway; and
placing the compression element over a proximal portion of the first contact element and a proximal portion of the second contact element, wherein the compression element is configured to move the first contact element and the second contact element towards each other such that a space between the first airway and the second airway is compressed.

23. The method of claim 22, further comprising crimping the contact element from an expanded configuration to a compressed configuration.
